(19) United States
(12) Patent Application Publication
(10) Pub. No.: US 2016/0017707 A1
(43) Pub. Date: Jan. 21, 2016

(54) Water Volume Fraction of Flowing Fluids

(71) Applicant: Schlumberger Technology Corporation, Sugar Land, TX (US)

(72) Inventors: Mikhail Stukan, Al-Khobar (SA); Mustapha Abbad, Al-Khobar (SA); Stephen John Remane Dyer, Dhahran (SA)

(21) Appl. No.: 14/334,843

(22) Filed: Jul. 18, 2014

(51) Int. Cl. E21B 49/08; G01N 27/22; G01N 33/18; G01N 27/04

(52) U.S. Cl. CPC E21B 49/08; G01N 27/04; G01N 27/22; G01N 33/18

(57) ABSTRACT

A measurement tool having one or more measurement cells disposed in a fluid flow path is provided. Measurements are made in each of the measurement cells and a fluid type is identified based on the outcome of the measurements. A numerical value is associated with each measurement cell based on the identified fluid type, and a total fractional volume of the identified fluids is determined using the associated numerical values. There may be a relatively large number of measurement cells distributed so as to substantially cover the cross-sectional area of the flow path, relatively few measurement cells distributed randomly in fixed locations in the flow path, or a single measurement cell moveably located in the flow path. The measurement cells measure a property such as resistivity, capacitance, dielectric constant, or electrical impedance. The total fractional volumes of the identified fluids may be determined using a statistic.

### BACKGROUND

With advances in drilling technology over the past thirty years, the use of horizontal and multilateral wells to develop reservoirs has become commonplace. The need to produce efficiently, economically, and environmentally-friendly has prompted the development of extended reach horizontal and multilateral wells that provide greater reservoir contact. However, increased wellbore lengths have led to certain production problems. For example, higher external pressure around the heel section of a wellbore resulting from a pressure drop due to frictional fluid flow in the wellbore causes a non-uniform fluid influx along the length of the wellbore and higher production rates at the heel. This often leads to early break-through (coning) of water or gas, which causes a reduction in oil recovery and an uneven sweep of the drainage area. To overcome this problem, Inflow Control Valves (ICVs) have been increasingly used in well completion to control and optimize reservoir production. Based on an accurate water volume fraction measurement downhole, a reservoir engineer may decide whether an ICV should be fully closed, fully open, or at some intermediate position.

When a mixture of oil and water is flowing in production tubing, determining the water volume fraction (also known as the “water cut” or “water content”) downhole can be done in several ways, each of which may involve different physics. Common techniques measure the electrical resistivity or capacitance of the mixture. Those parameters are generally proportional to the water content. However, it is known that a resistivity measurement can provide accurate water cut measurement if the water cut is in the 50%-100% range, while a capacitance measurement provides accurate water cut measurement if the water cut is in the 0%-30% range. Even if both of those measurement types are used, an estimate for the case in which the water cut is in the 30%-50% range may be inaccurate since making the estimate relies on modeling and extrapolation. Errors may arise, for example, due to the complex dynamics of the mixture (e.g., phase inversions may occur).

### SUMMARY

A measurement tool having one or more measurement cells disposed in a fluid flow path is provided. Measurements are made in each of the measurement cells and a fluid type is identified based on the outcome of the measurements. A numerical value is associated with each measurement cell based on the identified fluid type, and a total fractional volume of the identified fluids is determined using the associated numerical values. There may be a relatively large number of measurement cells distributed so as to substantially cover the cross-sectional area of the flow path, relatively few measurement cells distributed randomly in fixed locations in the flow path, or a single measurement cell moveably located in the flow path. The measurement cells measure a property such as resistivity, capacitance, dielectric constant, or electrical impedance. The total fractional volumes of the identified fluids may be determined using a statistic.

This summary is provided to introduce a selection of concepts that are further described below in the detailed description. This summary is not intended to identify key or essential features of the claimed subject matter, nor is it intended to be used as an aid in limiting the scope of the claimed subject matter.

### BRIEF DESCRIPTION OF THE DRAWINGS

The present disclosure is understood from the following detailed description when read with the accompanying figures. It is emphasized that, in accordance with the standard practice in the industry, various features are not drawn to scale. In fact, the dimensions of the various features may be arbitrarily increased or reduced for clarity of discussion. Embodiments of determining are described with reference to the following figures. The same numbers are generally used throughout the figures to reference like features and components.

FIG. 1A is a schematic drawing showing a grid of resistivity cells, with a set of example cells comprising the grid delineated, in accordance with the present disclosure.

FIG. 1B is a schematic drawing showing an enlargement of the set of delineated example cells of FIG. 1A, in accordance with the present disclosure;

FIG. 1C is a circuit diagram showing one embodiment of a resistance-based probe circuitry, in accordance with the present disclosure;

FIG. 2A is a schematic drawing showing an arrangement of capacitance cells, in accordance with the present disclosure;

FIG. 2B is a schematic drawing showing an enlargement of an example capacitance cell of FIG. 2A, in accordance with the present disclosure;

FIG. 2C is a circuit diagram showing one embodiment of a capacitance-based probe circuitry, in accordance with the present disclosure;

FIG. 3 is a schematic drawing showing randomly placed cells, in accordance with the present disclosure;

FIG. 4 is a schematic drawing showing a randomly located remote probe (in three different positions) used in conjunction with a detector, in accordance with the present disclosure;

FIG. 5 is a schematic drawing showing a mesh of measurement knots, in accordance with the present disclosure; and

FIG. 6 is a flowchart for at least one workflow embodiment, in accordance with the present disclosure.

### DETAILED DESCRIPTION

It is to be understood that the following disclosure provides many different embodiments, or examples, for implementing different features of various embodiments. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact.

Some embodiments will now be described with reference to the figures. Like elements in the various figures may be referenced with like numbers for consistency. In the following description, numerous details are set forth to provide an understanding of various embodiments and/or features. However, it will be understood by those skilled in the art that some embodiments may be practiced without many of these details and that numerous variations or modifications from the described embodiments are possible. As used here, the terms “above” and “below”, “up” and “down”, “upper” and “lower”, “upwardly” and “downwardly”, and other like terms indicating relative positions above or below a given point or element are used in this description to more clearly describe certain embodiments. However, when applied to equipment and methods for use in wells that are deviated or horizontal, such terms may refer to a left to right, right to left, or diagonal relationship, as appropriate. It will also be understood that, although the terms first, second, etc. may be used herein to describe various elements, these elements should not be limited by these terms. These terms are used to distinguish one element from another.

The terminology used in the description herein is for the purpose of describing particular embodiments and is not intended to be limiting. As used in the description and the appended claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event.]” or “in response to detecting [the stated condition or event],” depending on the context.

A system and method to measure the water cut in the full range 0-100% is disclosed. A statistical approach may be used to estimate the water content. Upon making a large number of local measurements on the system, for which the outcome of each experiment is either “water” or “oil”, the ratio of successful measurements (e.g., outcome is “water”) to the total number of measurements can lead to an estimate of the water cut. There are a number of ways to perform such statistical measurements on the flowing fluids. For example, the presence of water in the production tubing can be detected by measuring the electrical resistivity, the capacitance, the dielectric constant, or the electrical impedance in different portions of the production tubing. Those measurements can be performed in a completely random manner or using a grid arrangement. Upon taking the measurements, the probes at a particular measurement site indicate whether the fluid at that particular measurement site at the time of taking the measurement should be characterized as water or oil (or more generally, non-water). Taking the measurements together, a statistical approach may be used to determine the actual water cut, such as by averaging the individual probe indications.

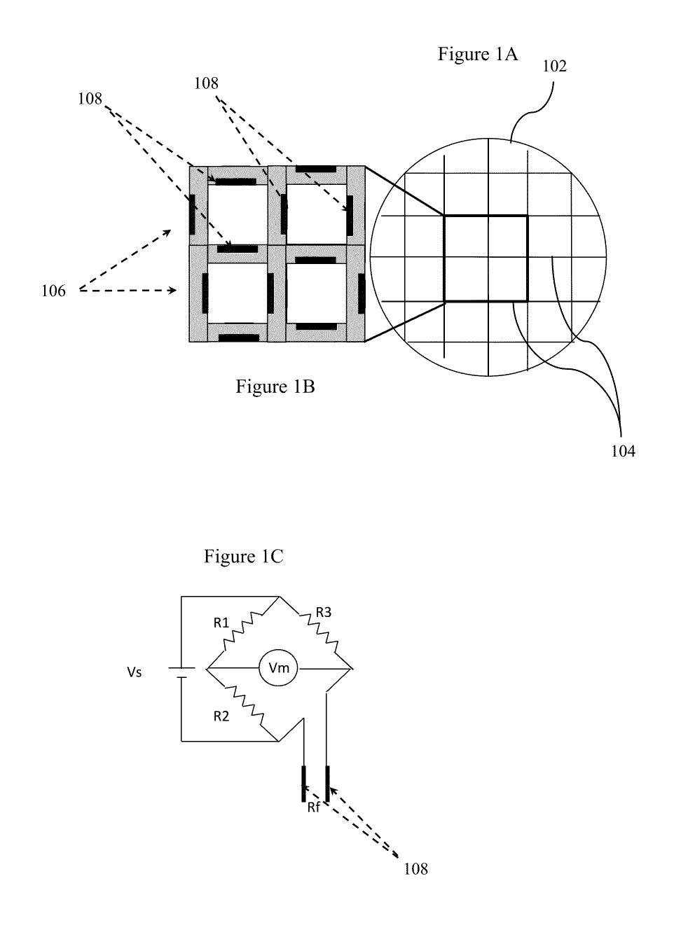

FIGS. 1A and 1B show a resistivity measurement tool 102 having cells 104, wherein each cell 104 includes a resistivity probe 106. In this particular embodiment, the cells are arranged to form a grid, and each probe comprises two resistivity poles 108. If the probe poles 108 are oriented “vertically” in a given cell 104, they are oriented “horizontally” in adjacent cells 104 to minimize cross-talk and interference.

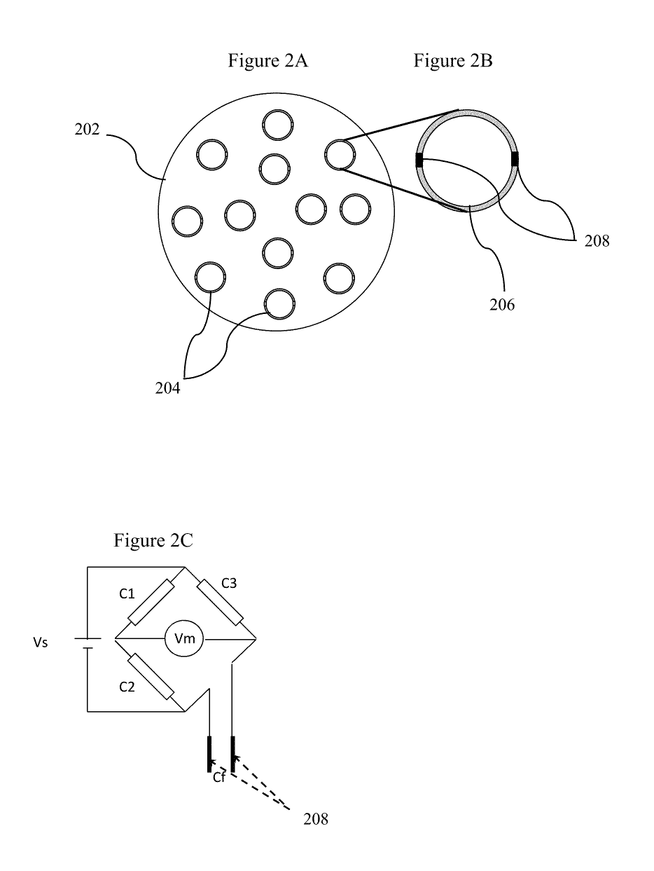

Similarly, FIGS. 2A and 2B show a capacitance measurement tool 202 having several cells 204 that include capacitance probes 206. Each capacitor probe 206 has two capacitor poles (plates) 208. When circular cells 204 are used, as shown, the alternating orientations in probe pole directions described above is not needed. Both of the configurations shown in FIGS. 1A, 1B, 2A, and 2B can be used to measure resistance or capacitance. In addition, as stated above, other probe types sensitive to other properties may be used.

Each tool 102/202 contains some desired number of cells 104/204 and probes 106/206. The diameter of the tool 102/202, the dimensions of the cells 104/204, and the number of probes 106/206 are known by design. The probe circuitry may be of various designs. For example, the circuitry may be patterned after the well-known Wheatstone Bridge, as shown in FIGS. 1C and 2C. The probes 106/206 in each cell 104/204 are energized by a given alternating voltage, either from the surface via cables or downhole by means of a downhole power generator or battery (with appropriate ac/dc converter circuitry). The frequency and amplitude of the supplied signal may be chosen by the operator. The resulting voltage measured across the bridge allows one to determine the unknown desired resistance or capacitance value. If a cell 104/204 is invaded mostly by a conductive fluid (water, brine, etc.) the resistance, Rf, should be very low, and that will be reflected in the measured voltage value, Vm. However, if the cell 104/204 is mostly invaded by oil, the resistance, Rf, will be very high and the measured voltage value will reflect that accordingly. Similarly, the high contrast between the capacitance in oil versus the capacitance in water enables the use of the capacitance-based circuit to detect water or oil in each cell 104/204.

When fluid invades a given cell 104/204, the fluid type is identified by the probe 106/206 based on the measured parameter (e.g., resistivity of water versus resistivity of oil). If the size of the cell 104/204 is sufficiently small, the fluid in the invaded cell 104/204 may be considered to be 100% of a particular fluid type (e.g., 100% water or 100% oil), which means that particular type of fluid occupies the total volume of that particular cell 104/204.

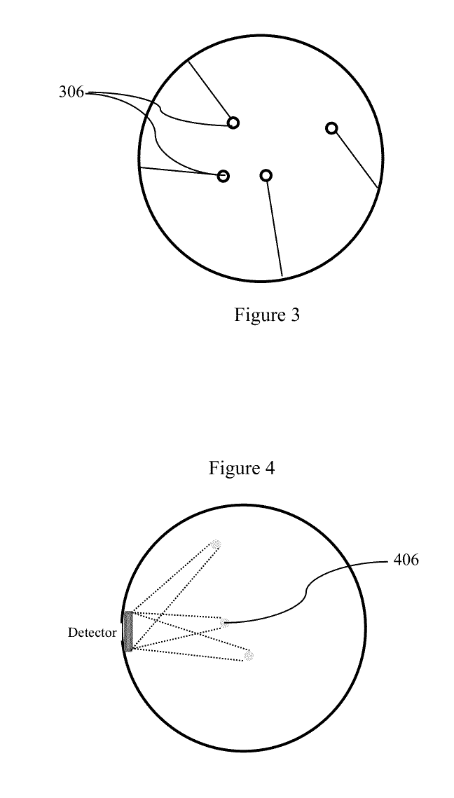

Two alternative configurations are shown in FIGS. 3 and 4, respectively. In both cases, water/oil detection is performed in a random way. However, in the case illustrated in FIG. 3, the probes 306 have permanent positions, while in the case shown in FIG. 4, the position of the measurement probe 406 is chosen randomly (shown in three locations, but is not so limited). Note that to prevent interference of the electrical signal from a given invaded resistivity cell from an adjacent, non-invaded resistivity cell, the positions of the resistivity probes 106 are such that the probes are vertical in one cell and horizontal in an adjacent one. This configuration is not needed in the embodiment in which capacitance measurements are used. For that tool, the cells may be circular and placed relatively far from one another.

One statistical approach used to compute the actual water cut in the production tubing involves determining a water cut average over the grid. That is, the sum of the individual responses in the cells, wherein each response is characterized as a zero or a one, is divided by the total volume of the grid. To increase precision, the measurements may be averaged over time.

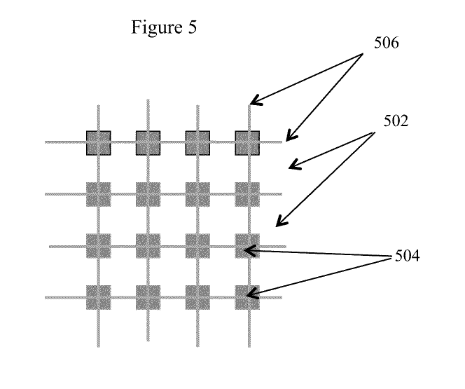

In at least some of the embodiments shown, the measurements cover the full flow cross sectional area of the tubular (e.g., the grid shown in FIG. 1A or the array of holes shown in FIG. 2A). In this way one can obtain complete information about the flow and try to ensure the measurements are free from bias. At least some prior art techniques rely on time-averaging and can provide biased results in the case of structured flows. While embodiments described above contemplate placing the measuring device in a tubular or across the borehole, the measuring device may alternatively be installed just behind the sand screen to measure the water cut of the fluid coming into the production tubing. The grid can be constructed as shown in FIG. 1A, where measurements are performed for each individual cell, or it can be arranged as shown in FIG. 5, in which measurements are performed in the “knots” 502 of the mesh.

For the embodiment shown in FIG. 5, the reading of information about the phase in each knot 502 can be done in a manner similar to that done for magnetic-core memory. In all cases the response from each of the knots is a zero or one (i.e., water phase or non-water phase), and that value may be read via a connecting net. The total water cut may be determined by computing a simple average: the total number of ones divided by the total number of knots. This may be also be done for each line of knots to get a water cut estimate for any given line (i.e., total number of ones for knots in a given line divided by the total number of knots in that line).

The approaches illustrated in FIGS. 3 and 4, instead of measuring full cross-sectional areas, rely on the randomness of the measurements. That is, measurements are performed randomly in different places of the flow cross-sectional area (with a uniform distribution of possible measurement locations). The result of each measurement again will be either a one or zero. As before, the water cut can be calculated for this case by computing a simple average.

While embodiments described herein are described in terms of downhole production tubing, measurements may also be made on fluids in surface tubing and the water cut may be determined for the fluids flowing in that tubing as well.

The addition of electrostatic impulses to a fluid mixture can be used to aid in the separation of that mixture. This embodiment could be used in a downhole (or surface) device or pipeline to aid in the separation of an oil-water emulsion. The separation into an emulsion generally reduces the fluid viscosity, resulting in an improved ability to pump the fluids using, for instance, downhole cavitation pumps (also known as electrical submersible pumps or ESPs).

Attention is now directed to processing procedures, methods, techniques, and workflows that are in accordance with some embodiments. Some operations in the processing procedures, methods, techniques, and workflows disclosed herein may be combined and/or the order of some operations may be changed. It is important to recognize that geologic interpretations, sets of assumptions, and/or domain models may be refined in an iterative fashion. This concept is applicable to the processing procedures, methods, techniques, and workflows discussed herein. This iterative refinement can include use of feedback loops executed on an algorithmic basis, such as at a computing device and/or through manual control by a user who may make determinations regarding whether a given step, action, template, or model has become sufficiently accurate.

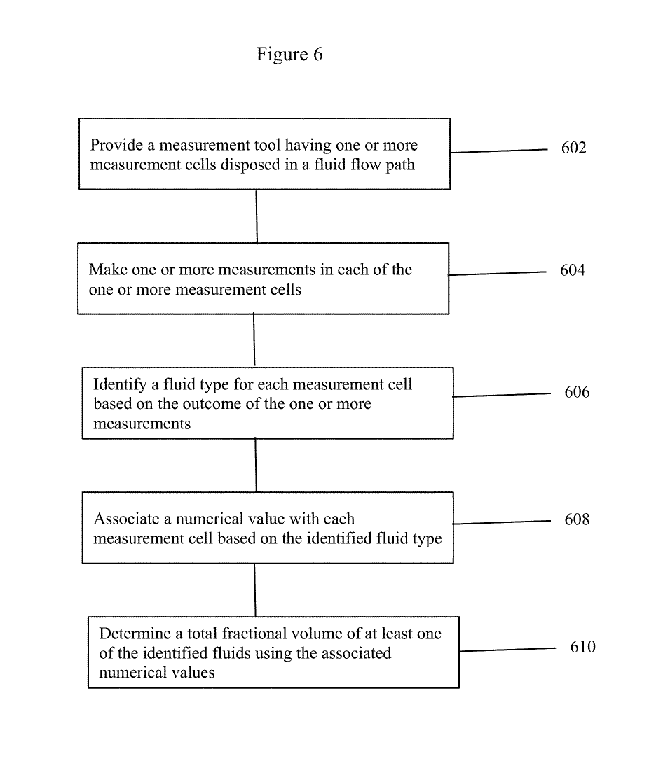

FIG. 6 shows a flowchart illustrating an embodiment in accordance with this disclosure. In this embodiment, the workflow comprises providing a measurement tool having one or more measurement cells disposed in a fluid flow path (602). One or more measurements are made in each of the one or more measurement cells (604). A fluid type is identified for each measurement cell based on the outcome of the one or more measurements (606). A numerical value is associated with each measurement cell based on the identified fluid type (608) and a total fractional volume of at least one of the identified fluids is determined using the associated numerical values (610).

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the scope of the present disclosure, and that they may make various changes, substitutions, and alterations herein without departing from the scope of the present disclosure.

The Abstract at the end of this disclosure is provided to comply with 37 C.F.R. §1.72(b) to allow the reader to quickly ascertain the nature of the technical disclosure. It is submitted with the understanding that it will not be used to interpret or limit the scope or meaning of the claims.

While certain embodiments have been set forth, alternatives and modifications will be apparent from the above description to those skilled in the art. These and other alternatives are considered equivalents and within the scope of this disclosure and the appended claims. Although only a few example embodiments have been described in detail above, those skilled in the art will readily appreciate that many modifications are possible in the example embodiments without materially departing from the present disclosure. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. §112, paragraph 6 for any limitations of any of the claims herein, except for those in which the claim expressly uses the words ‘means for’ together with an associated function.

What is claimed is:

1. A method, comprising:
providing a measurement tool having one or more measurement cells disposed in a fluid flow path;
making one or more measurements in each of the one or more measurement cells;
identifying a fluid type for each measurement cell based on the outcome of the one or more measurements;
associating with each measurement cell a numerical value based on the identified fluid type; and
determining a total fractional volume of at least one of the identified fluids using the associated numerical values.

2. The method of claim 1, wherein the fluid flow path is an interior region of a tubular and the one or more measurement cells comprise a relatively large plurality of measurement cells distributed so as to substantially traverse the cross-sectional area of the tubular.

3. The method of claim 1, wherein the fluid flow path is an interior region of a tubular and the one or more measurement cells comprise a relatively small plurality of measurement cells distributed randomly in fixed locations in the tubular.

4. The method of claim 1, wherein the fluid flow path is an interior region of a tubular and the one or more measurement cells comprise a single measurement cell moveably located in the tubular.

5. The method of claim 1, wherein the one or more measurement cells measure a parameter selected from the group consisting of resistivity, capacitance, dielectric constant, and electrical impedance.

6. The method of claim 1, wherein the numerical value is either a zero or a one.

7. The method of claim 1, wherein the one or more measurement cells comprises a pair of resistivity probes.

8. The method of claim 7, wherein the orientations of adjacent resistivity probes are orthogonal.

9. The method of claim 1, wherein the one or more measurement cells comprise a capacitor.

10. The method of claim 1, wherein the fluid flow path is substantially longitudinal within a downhole tubular, a surface tubular, or a wellbore; or substantially radial within the wellbore.

11. The method of claim 1, wherein the identifying a fluid type comprises selecting a fluid from the group consisting of water and non-water.

12. The method of claim 1, wherein the associating a numerical value based on the identified fluid type comprises correlating the one or more measurements to known related quantities.

13. The method of claim 1, wherein the determining a total fractional volume comprises computing an average.

14. The method of claim 1, wherein the determining a total fractional volume comprises dividing the number of measurements cells for which the identified fluid type is a particular fluid type by the total number of measurement cells.

15. The method of claim 1, wherein the one or more measurements made by a particular measurement cell comprise a plurality of measurements, and further comprising:
computing an average of the plurality of measurements for that particular measurement cell; and
using the computed average as a representative measurement for that particular measurement cell.

16. A system, comprising: one or more measurement cells disposed in a fluid flow path.

17. The system of claim 16, wherein the one or more measurement cells are arranged to form a grid or mesh.

18. The system of claim 16, wherein the one or more measurement cells are randomly distributed.

19. The system of claim 16, wherein the one or more measurement cells comprise lines of knots.

20. A method, comprising:
applying one or more electrostatic impulses to a fluid mixture to separate the fluid mixture into an emulsion;
reducing the viscosity of the fluid by forming the emulsion; and
pumping the reduced viscosity fluid.
